(19) United States
(12) Patent Application Publication
(10) Pub. No.: US 2023/0330355 A1
(43) Pub. Date: Oct. 19, 2023

(54) Single-Use Adapter Attached to Drug/Vaccine Vials Compatible with Hypodermic Needles to Enable Injection

(71) Applicant: Becton Dickinson Holdings Pte. Ltd., Singapore (SG)

(72) Inventor: Guan Bin Lee, Singapore (SG)

(21) Appl. No.: 18/035,116

(22) PCT Filed: Nov. 2, 2021
(86) PCT No.: PCT/SG2021/050664

§ 371 Date: May 3, 2023

(30) Foreign Application Priority Data
Nov. 4, 2020 (SG) 10202010968P

(51) Int. Cl. A61M 5/34; A61M 5/142; A61M 5/24

(52) U.S. Cl. CPC A61M 5/345; A61M 5/1424; A61M 5/2466; A61M 11/007

(57) ABSTRACT

An adapter for attaching a needle to a pump actuated vial for injection of a vaccine or drug using a syringe-type injection motion while eliminating the time-consuming preparation of the injection device. The adapter includes a spray pump device having a first end defining a luer tip and a second end configured for accessing a media reservoir to receive a metered dose of flowable media. The luer tip is configured to be secured to a needle cannula to deliver the metered dose of the flowable media received from the spray pump device.

### CROSS-REFERENCE TO RELATED APPLICATION

The present application claims priority to Singapore Application No. 10202010968P, filed Nov. 4, 2020, entitled “Single-Use Adapter Attached to Drug/Vaccine Vials Compatible with Hypodermic Needles to Enable Injection” the entire disclosure of which is hereby incorporated by reference in its entirety.

### BACKGROUND OF THE INVENTION

### Field of the Invention

The present invention relates to an adapter for attaching a needle to a pump actuated vial for injection of a vaccine or drug, and more particularly relates to an adapter for immediate activation and administration of drugs/vaccines using a typical syringe-type injection motion while eliminating time-consuming preparation of the injection device.

### Description of Related Art

Immunization of individuals has long been a known technique for controlling diseases such as measles, mumps, rubella, polio, hepatitis, chicken pox, and the like. Immunizations or vaccines are typically applied using syringes, which include the step of drawing out a dose of the drug/vaccine from a vial and into the syringe prior to administration of the injection. This can be a time-consuming task and requires a certain amount of medical training to ensure that the dosage is correct. Oftentimes, especially in developing countries, vaccines are administered to a large number of individuals over a short period of time. Accordingly, elimination of the step of drawing the medication into the syringe would be desirable.

Pre-filled vials, such as shown in U.S. Pat. No. 5,554,125, eliminates the step of drawing the medication into the syringe. The drawbacks to the use of a pre-filled vial is that it requires the health care worker to possess a particular type of syringe to use the pre-filled vial and to know how to assemble the vial within the syringe. This assembly can also be time-consuming, especially when vaccinating a large number of people. Pre-filled syringes are also known and used as time-saving devices as the entire syringe/pre-filled vials are preassembled and shipped as an entire unit, however, these devices are expensive, require a large packaging footprint to accommodate the plunger rod, and require special packaging to prevent inadvertent actuation of the plunger rod.

One economical replacement to pre-filled syringes is taught by U.S. Pat. No. 4,955,871, which includes a collapsible reservoir pre-filled with a single dose of medication in fluid communication with a needle. The reservoir is formed from a pair of opposed flexible membranes which are sealed along their edges to enclose the medication therein. Application of a pinching or squeezing force to the opposed membranes causes the membranes to collapse, which, in turn, causes the medication to exit the reservoir and into the patient via the needle. One difficultly encountered using this type of device is that it requires a certain amount of dexterity to operate, requires careful handling to prevent inadvertent actuation, requires a certain amount of hand strength to apply enough force to expel all of the medication, and requires a specific type of packaging must be used to ensure that the contents are not inadvertently released during shipment.

Spray pump devices for delivering medicine into nasal passages are known. Examples of different types of spray pump devices are disclosed in U.S. Patent Application No. 2012/0193377, U.S. Pat. Nos. 5,893,486, and 7,299,949, their disclosures of which are incorporated by reference in their entirety.

There is a need in the art for a system and method for the immediate activation and administration of drugs/vaccines which can be easily manipulated using a typical syringe-type injection motion while eliminating time-consuming preparation of the injection device.

### SUMMARY OF THE INVENTION

The present disclosure is also directed to an adapter for delivering a metered dose of flowable media. The dose of flowable media can be applied subcutaneously or intramuscular. The adapter includes a spray pump device having a first end defining a luer tip and a second end configured for accessing a media reservoir to receive a metered dose of flowable media. The luer tip is configured to be secured to a needle cannula to deliver the metered dose of the flowable media received from the spray pump device.

The present disclosure is also directed to a device for delivering a metered dose of flowable media. The device includes a media reservoir configured for holding a flowable media and an actuator having a first end, a second end, and a conduit extending between the first and second end, said second end of said actuator configured for movement between a first position and a second position upon the application of an actuating force thereto. An inlet is provided at the second end of the actuator. The inlet is configured for transferring a metered dose of the flowable media from the media reservoir into the conduit of the actuator. An opening is provided in the first end of the actuator and a connecting member is associated with the first end of the actuator. The connecting member is configured for cooperation with a needle cannula such that the needle cannula is in fluid communication with the opening in the first end of the actuator to deliver the metered dose of the flowable media.

According to one arrangement, the connecting member can comprise a Luer Slip Tip. The use of a Luer Slip Tip enables a quicker turnaround time per injection action than Luer Lok Tips. However, it can be appreciated that other connecting members, including Luer Lok Tips can be used with the adapter of the invention. The needle cannula can be preassembled to the luer tip and packaged as a complete unit. Alternatively, the needle cannula can be assembled onto the actuator at the time of injection. A removable cover for the needle cannula can be provided.

According to one embodiment, the media reservoir can be a removable vial containing a single-use dose of the flowable media. The vial can contain a single dose or multiple doses, dedicated to a single patient, or for multiple patients with the use of a series of single-use delivery devices. The vial can include a pierceable septum and the second end of the actuator can include a piercing element associated therewith. This piercing element includes a fluid passageway for providing fluid communication between the actuator and vial. According to one embodiment, the piercing element can comprise a non-patient cannula.

The actuator can include a biasing element to return the actuator to the first positon after application of the actuating force thereto. This biasing element can be a spring or any other well-known biasing element.

The device can also include a valve configured for cooperation with the inlet of the actuator, wherein movement of the actuator to the second position causes said valve to open to admit the metered dose of the flowable media into the conduit of the actuator.

The present disclosure is also directed to a method of delivering a metered dose of flowable media. The method includes providing a media reservoir having a spray pump device associated therewith, the spray pump device having an exit opening and being configured for supplying a metered dose upon application of an actuation force thereto. The method further includes securing a needle cannula to the exit opening of the spray pump device, wherein application of the actuation force to the spray pump device causes the metered dose of flowable media to exit through the needle cannula.

According to one embodiment, a luer tip, such as a Luer Lok Tip, can be added to the exit opening of the spray pump device for securing the needle cannula thereto.

The media reservoir can be located within the pump device itself or can be a separate vial. The separate vial can have a pierceable septum and the spray pump device can include a piercing element configured for piercing the septum. In this arrangement, the method includes piercing the septum of the vial with the piercing element. According to one embodiment, the piercing element can comprise a non-patient cannula. At least one of the vial and the spray pump device includes a connecting assembly to secure the vial and the spray pump device together. This connecting assembly can comprise a threaded assembly, a clip, a latch, and the like.

A removable cover can be provided for the needle cannula and a collapsible sleeve can be provided for the piercing element to maintain sterility of the needle cannula and/or piercing element.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a cross-sectional view of the device for delivering a metered dose of flowable media, in accordance with an embodiment of the present invention.

FIGS. 2A-2D show certain individual components of the device of FIG. 1 in accordance with an embodiment of the present invention.

FIG. 3 shows a cross-sectional view of an adapter for delivering a metered dose of flowable media in accordance with an embodiment of the present invention.

FIG. 4 shows a cross-sectional view of one embodiment of a spray pump device that can be used with the adapter in accordance with an embodiment of the present invention.

FIGS. 5A and 5B show a front perspective and cross-sectional view of another embodiment of a spray pump device that can be used with the adapter in accordance with an embodiment of the present invention.

FIG. 6 shows a cross-sectional view of another embodiment of a spray pump device that can be used with the adapter in accordance with an embodiment of the present invention.

FIG. 7 shows a cross-sectional view of still another embodiment of a spray pump device wherein a cutting element is incorporated in the spray pump device which can be used with the adapter in accordance with an embodiment of the present invention.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

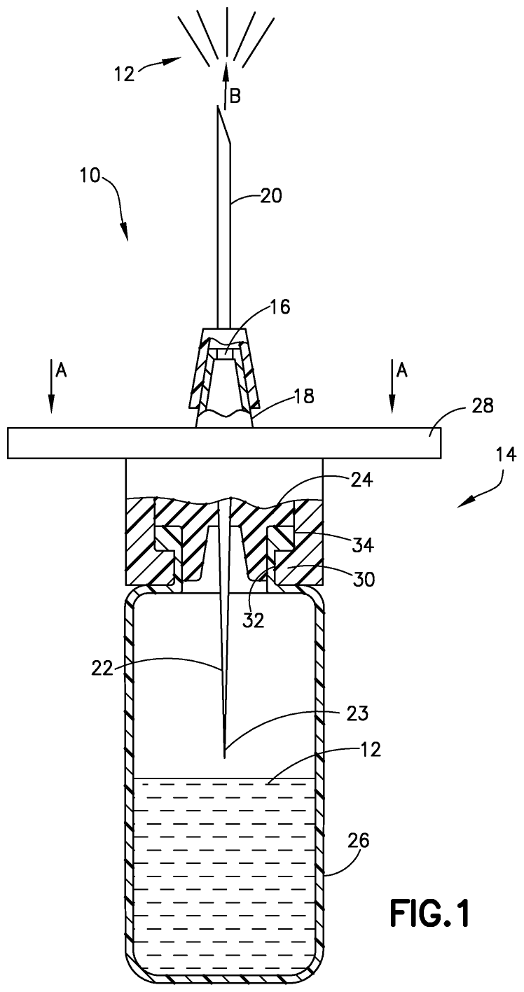

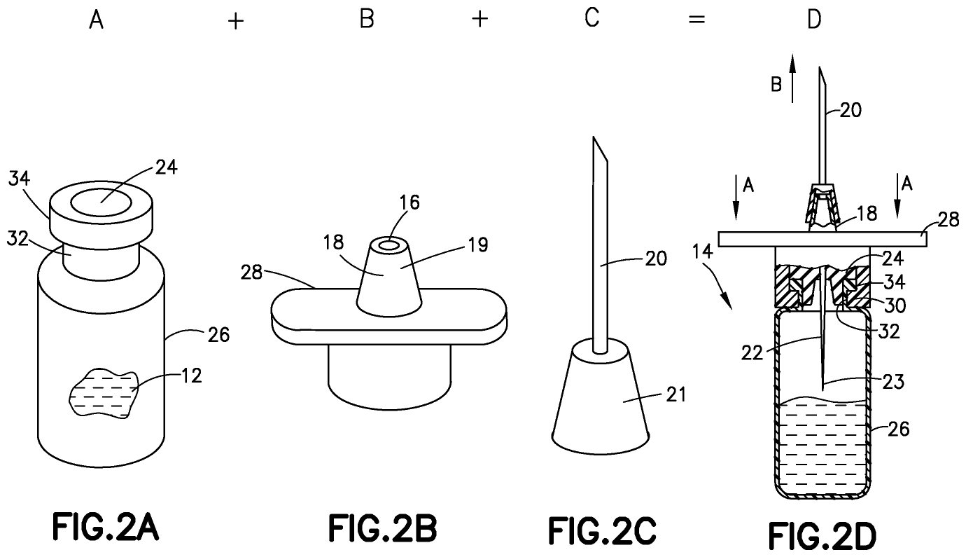

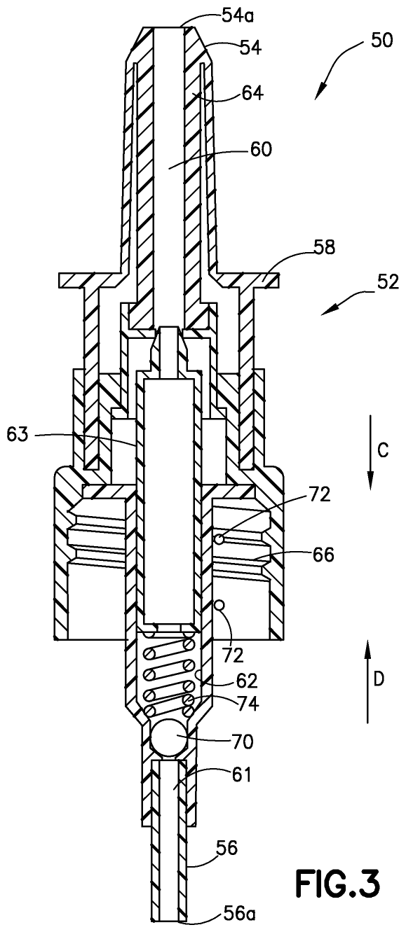

Reference is now made to FIG. 1 which illustrates a perspective view of the device, generally indicated as 10, for delivering a metered dose of flowable media 12. The dose of flowable media can be applied subcutaneously or intramuscular. Reference is also made to FIGS. 2A-2D which show the individual components of the device 10. The device includes a spray pump device, generally indicated as 14, having an exit opening 16. A first end 18 of the spray pump device 14 is configured for cooperating with a needle cannula 20. According to one embodiment, the first end 18 of the spray pump device 14 can include a luer tip 19 and the needle cannula 20 includes a needle base 21 configured for latching onto the luer tip 19. The spray pump device 14 has a second end 22 that can include a piercing or cutting element 23, such as a non-patient needle cannula, that is capable of piercing a septum 24 of a vial 26 containing the media 12. Application of an actuation force, as shown by arrow A, such as by applying pressure to flange 28, causes movement of the spray pump device from a first direction to a second direction such that a metered dose of flowable media 12 enters into the spray pump device 14. Release of the actuation force causes the spray pump device to move back to the first position as shown by arrow B causing the metered dose to exit through the exit opening 16 into the needle cannula 20, and subsequently into a patient. The spray pump device can be secured to the media vial by a clip-on arrangement, as illustrated by 30, 32, and 34, a threaded arrangement, a snap fit arrangement, or any other well-known arrangement.

According to one embodiment, the media reservoir or vial 26 can contain a single-use dose of the flowable media 12. Alternatively, the vial 26 can include multiple doses for use with a series of single-use delivery devices or spray pump devices 14.

Reference is now made to FIG. 3 which shows a cross-sectional view of an adapter, generally indicated as 50, for delivering a metered dose of flowable media including one type of spray pump device 14 that can be used with the device 10 of the present application. The spray pump device 14 comprises an actuator, generally indicated as 52, having a first end 54, a second end 56, and a conduit 60 extending between the first end 54 and the second end 56. The second end 56 of the actuator is configured for movement between a first position and a second position upon the application of an actuating force thereto, as shown by arrow C. This actuating force can be achieved by the application of a distally directed force to flange 58. An inlet 56a is provided at the second end 56 of the actuator 52. The inlet 56a is configured for transferring a metered dose of the flowable media from the media reservoir, as shown by 26 in FIG. 1, into the conduit 60 of the actuator 52. The second end 56 of the actuator 52 can comprise a piercing element, such as a non-patient cannula that includes a fluid passageway 61 for providing fluid communication between the actuator 52 and the vial 26 via a suction chamber 62 and a vertical bore 63. The adapter 50 can be secured to the media reservoir or vial 26 by a threaded engagement, such as shown by threads 66, by the snap-fit arrangement 30, 32, 34 as shown in FIG. 1, or by any other known attachment arrangement. An opening 54a is provided in the first end 54 of the actuator 52 and a connecting member 64, which can be a luer or any other well-known connecting member, is associated with the first end 54 of the actuator 52. The connecting member is configured for cooperation with a needle cannula 20, as shown in FIG. 1, such that the needle cannula is in fluid communication with the opening Ma in the first end of the actuator 52 to deliver the metered dose of the flowable media.

With continuing reference to FIG. 3, the adapter 50 can also include a valve, such as a ball seal valve 70, configured for cooperation with the inlet 56a and passageway 61 of the actuator, wherein movement of the actuator to the second position causes said valve 70 to open to admit the metered dose of the flowable media into the conduit 60 of the actuator. It can be appreciated that in the embodiment shown in FIG. 3, the conduit 60 comprises the passageway 61, suction chamber 62, and vertical bore 63, all of which are in fluid communication with one another to transfer the flowable media from the media reservoir 26 through the actuator 52 and out of opening 54a. The actuator 52 can also include air replacement pores 72 for regulating pressure within the actuator 52 during use of the device. The actuator can include a biasing element 74 to return the actuator 52 to the first positon, as shown by arrow D, after application of the actuating force thereto. This biasing element can be a spring or any other well-known biasing element.

It can be appreciated that the needle cannula 20 can be preassembled to the luer tip 19 or connecting member 64 and packaged as a complete unit. Alternatively, the needle cannula 20 can be assembled onto the actuator 52 at the time of injection. A removable cover (not shown) for the needle cannula can be provided as well as a collapsible sleeve (not shown) for the piercing element to maintain sterility of the needle cannula and/or piercing element.

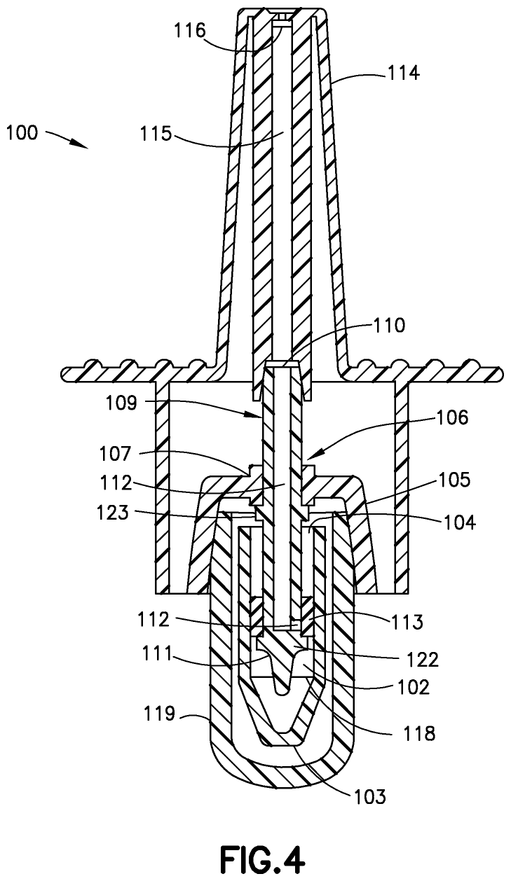

Reference is now made to FIG. 4 which shows a cross-sectional view of one embodiment of a spray pump device, generally indicated as 100 that can be used with the adapter 50, shown in FIG. 3. The nasal spray pump 100 includes a reservoir 102 configured for holding a single dose of material (not illustrated) to be dispensed. The reservoir 102 extends between a closed end 103 and an open end 104. A cap 105 is configured to be disposed over the open end 104 of the reservoir 102. The cap 105 includes an aperture 106 centrally disposed on an upper surface 107 of the cap 105. A stem 109 is configured to be retained in the aperture 106 of the cap 105 when the cap 105 is disposed over the open end 104 of the reservoir 102. The stem 109 is also configured for sliding movement through the aperture 106 of the cap 105 between a first stem position (as shown in FIG. 4) wherein a piston 113 is disposed in the reservoir 102 in the first piston position and a second stem position (not illustrated) wherein the stem 109 is moved toward the closed end 103 of the reservoir 102 where the piston 113 is moved from the first piston position to the second piston position (not illustrated). It will be appreciated that the piston 113 can alternatively sit on top of the reservoir open end 104 in other preferred embodiments if desired. The piston 113 needs to seal or substantially seal with both the reservoir 102 and the stem 109.

The nasal spray pump 100 includes a nasal spray pump head 114 configured to be received by the outlet end 110 of the stem 109. The nasal spray pump head 114 includes a path 115 through which material travelling through the outlet end 110 of the stem 109 can pass and exit at a nasal spray pump head aperture 116 configured to be disposed in or adjacent to a nasal cavity of a user to receive material dispensed therefrom.

A piston engagement means 118 is disposed within the reservoir 102 at a predetermined height above the closed end 103. The piston engagement means 118 extends the predetermined distance such that movement of the stem towards the second stem position causes the piston 113 to engage with the piston engagement means 118 to retain or retard movement of the piston 113 toward the closed end 103 of the reservoir 102. As the stem 109 is moved into the stem second position, the piston 113 engages with the piston engagement means 118 and the stem 109 is continued toward the stem second position with the piston 113 retained thereby exposing the conduit 112 inlet. The conduit 112 is then in communication with the material in the reservoir 102 and it is then able to flow therethrough.

As the stem is moved into the stem second position and the piston 113 engages the piston engagement means 118, movement of the stem 109 into the second stem position causes the material disposed in the reservoir 102 to be forced out under pressure through the conduit 112 to exit the outlet end 110 of the stem 109. The material then travels through path 115 in the nasal spray pump head 114 to exit at the nasal spray pump head aperture 116 for delivery to a nasal cavity (nostril) of a user. The adapter 50 of the present invention, can be secured to the spray pump head 114 so that the material can be delivered subcutaneously or intramuscularly to a patient.

A canister 119 is provided and the cap 105 is configured to be disposed over and seal the canister 119. The piston 113 is engaged about the stem 109 in an interference fit so as to provide resistance to movement of the piston 113 from the first piston position where the conduit 112 is sealed or substantially sealed by the piston 113. A lower shoulder 122 is disposed about the stem 109 to provide a seat or stop means to prevent the piston moving down the stem 109 toward the lower end 111. An upper shoulder 123 is disposed about the stem 109 and spaced apart from the lower shoulder 122 toward the outlet end 110 of the stem 109 to prevent movement of the stem 109 away from the closed end 103 of the reservoir 102, which limits the length of travel of the stem 109 within the cap 105 between the first stem position and the second stem position.

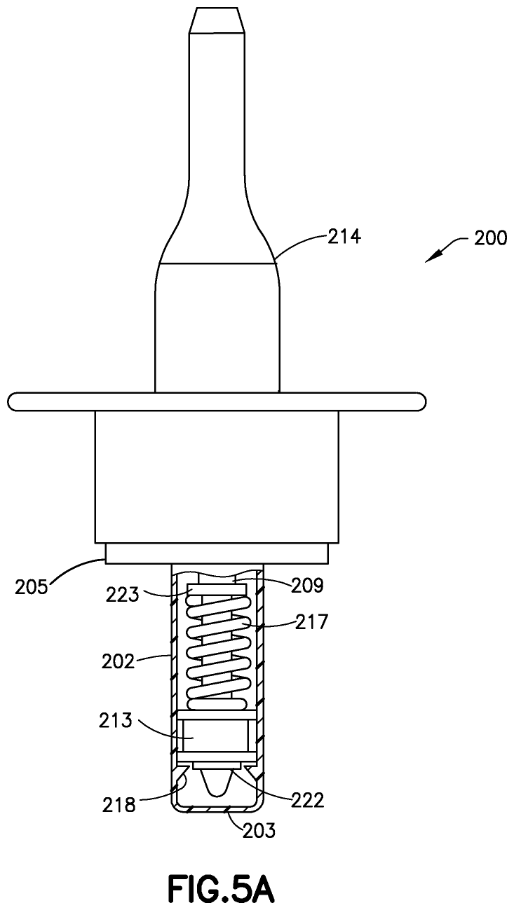

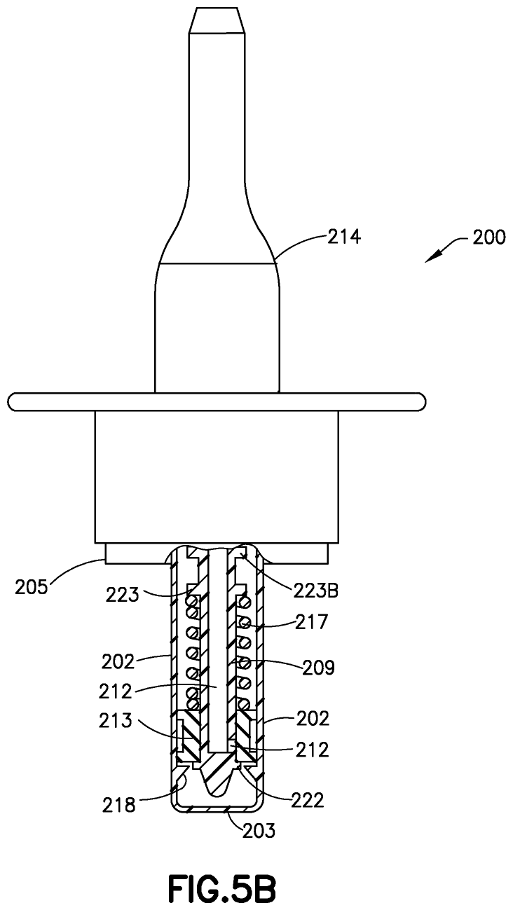

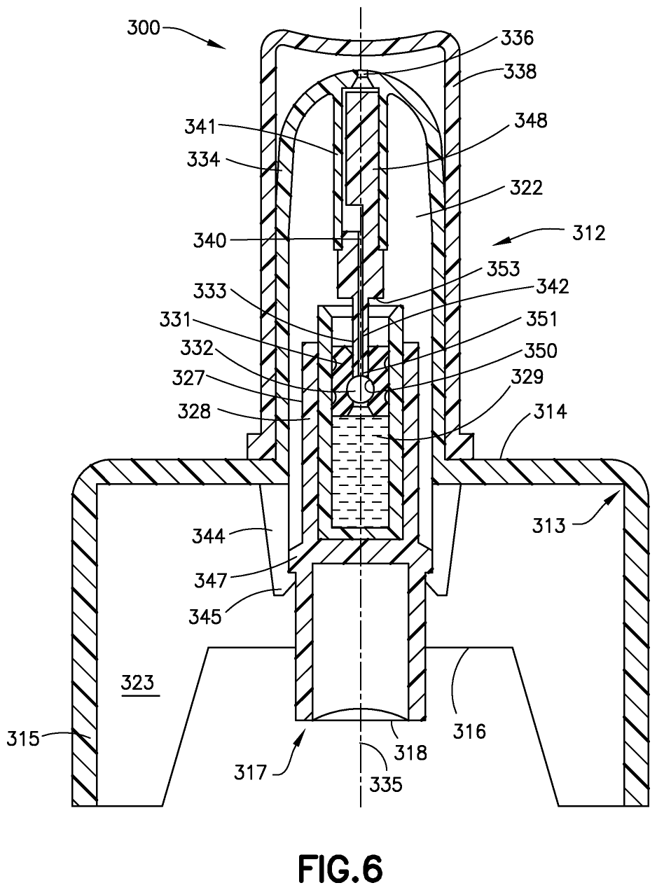

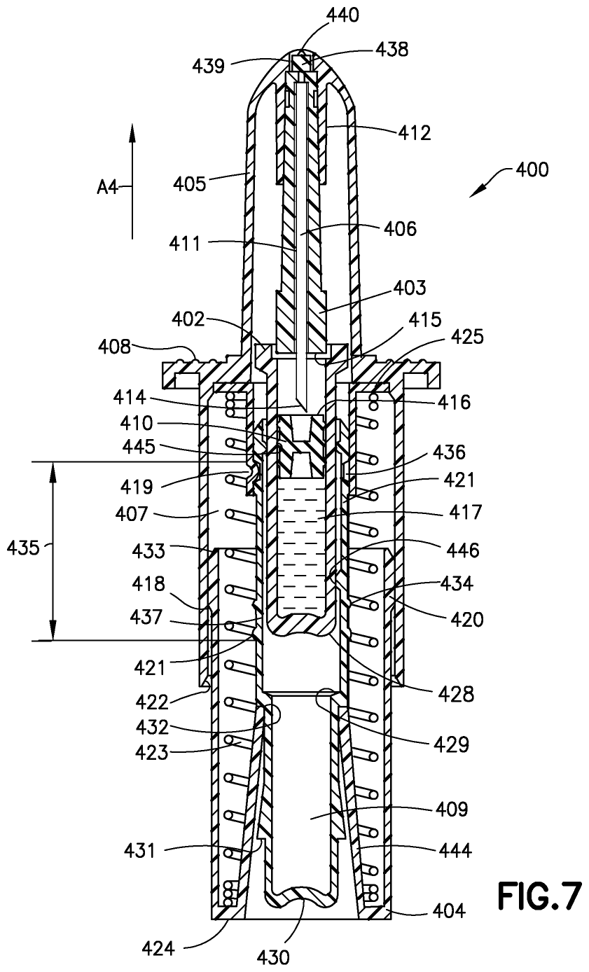

Referring now to FIGS. 5A and 5B, there is shown a nasal spray pump 200 according to an embodiment that can be used with the adapter 50 of the invention shown in FIG. 3. In this embodiment, the canister 119 of FIG. 4 is not used and the reservoir 202 sealingly engages with the collar 205. The piston 213 is resiliently biased into the first piston position, as shown in FIGS. 6 and 7. A slight interference fit between the piston 213 and stem 209 is also provided to provide bias against movement of the piston 213 into the second piston position. A compression spring 217 is disposed about the stem 209 to bias the piston 213 into the first piston position with a predetermined force. The spring 217 is disposed about the stem 209 intermediate the shoulder 223 and the piston 213. The piston 213 is engaged at a lower end at a lower shoulder 222.

As best seen in FIG. 5B a second upper shoulder 223B is spaced apart from the upper shoulder 223 toward an outlet end of the stem 209. The second upper shoulder 223B is retained within the collar 205.

Movement of the stem 209 toward the closed end 203 of the reservoir 202 into the second stem position causes the piston 213 to engage with the piston engagement means 218 to retain or retard movement of the piston 213 relative to the stem 209 whilst allowing downward movement of the stem 209. The spring 217 provides a bias against movement of the piston 213 from the first piston position to the second piston position.

As with the embodiment described in FIG. 4, movement of the stem 219 to the second stem position and movement of the piston 213 into the second piston position unseals or moves clear of the exit of the conduit 212 about which the piston 213 is disposed in the first piston position to allow material in the reservoir 202 to be dispensed through the conduit 212 and through the outlet end of the stem 209 for dispensing intranasally. It will be appreciated that piston engagement means 218 can be removed and the piston 213 moved against the bias of spring 217 under the influence of the pressure provided by movement of the stem 209 and/or piston 213 toward the closed end 203 of the reservoir 202.

The adapter 50 of the present invention can be secured to the nasal spray pump head 214 of FIGS. 5A and 5B to facilitate use of the nasal spray pump 200 and delivery of the contents through the cannula 20 to the patient. FIG. 6 shows yet another embodiment of a device 300 that can be used with the adapter 50 (FIG. 3) of the invention. The device 300 comprises a main body 312 which includes an actuator section 313 having two actuator shoulders 314 and a jacket 315 adjoining thereto. The jacket 315 incudes a cutout 316 at its two sides.

The main body 312 comprises further a port section 334 protruding upwards and including a spray nozzle 336. When not in use this port section 334 is covered by a protective sleeve 338.

The interior 322 of the port section 334 is in connection with the space 323 in the jacket. In this continuous interior 322, 323 a media reservoir 327 is accommodated that comprises a container open at one end, inserted in a corresponding mount 328 of an actuating sleeve 347. This mount 328 includes an actuating face 318.

The actuator sleeve 317 is disposed shiftably between mounting tabs 344 along the center line 335 of the device, these tabs being elastic and carrying at their ends counter—hooking latching projections 345 which snap into place behind the guide flange 347 and thus safeguard the actuator sleeve from dropping out after it being inserted in place from the bottom. At this position, defined break links forming an originality safeguard may be provided which are broken on actuation.

The media reservoir contains the medium 329 to be discharged, for example, a medicinal drug to be received through the skin. It is closed off by a flexible, sealing plunger plug 331 pressed into place, having an inner opening 333 into which a ball is pressed as a closure element 332. The orifices of the opening 333 may be flared.

This ball 332 is located in the portion of the opening 333 facing the medium. It is so large that it displaces the material of the plunger plug, which is relatively flexible, it thereby creating a seat 350 for itself. As a result of this the sealing pressure exerted by the outer surfaces of the plunger plug 332 on the inner walls of the media reservoir forming the pump barrel is increased.

The opening 333 of the plunger plug 331 is engaged by an actuator 342 which is formed as a tube cut off at the bottom more or less straight, but preferably configured with transverse recesses 351 in its orifice surface so as not to be closed off by the ball 332. The outer periphery of the actuator 342 is a seal-fit in the opening 333 of the plunger plug 331, its length being the same as that of the plunger plug in the example embodiment. Adjoining it is an actuating shoulder serving as a stop surface, this shoulder being provided on an actuator carrier 348. The latter is pressed into place in a downwards facing receiving sleeve 341 of the main body 313 and forms by its top face facing the nozzle 336 the nozzle vortex grooves affecting atomization. The actuator and the actuator carrier 348 integral therewith features a full-length discharge passage 340 which is formed in the portion of the actuator by a central inner passage of the port-type actuator and in the upper part by an outer fluid guidance groove.

The device 300 functions as follows. The media reservoir 327 filled with the medium 329 to be discharged and closed off by the plunger plug 331 and the closure element 332 is inserted in the actuator sleeve 317 and located from below centrally between the mounting tabs 344 until it latches into place as shown. As a result of this arrangement, the actuator 342 is advanced to a position just before, or ahead of the position from which the actuator begins the actuating movement which pushes out the closure element 332. When the discharge device is actuated it is held by two fingers locating the opposing actuator shoulders 314 whilst the thumb is used to press the actuator face 318. This causes the actuator sleeve 317 to be shifted upwards and the actuator 342 comes up against the ball 332 presenting the former with a counterpressure stemming, on the one hand, from the force pressing on the plunger plug 331 and, on the other, from the counterpressure of the usually incompressible medium 329. This is overcome by the actuator 342 forcing the ball further into the interior of the media reservoir, the plunger plug 331 being able to move slightly upwards accordingly or it experiencing a deformation to make room for the displaced fluid.

When the ball 332 is forced out of the full-length drilled opening 333 of the plunger plug and into the media reservoir the central discharge opening 340 is then free and the medium is able to move via the latter to the nozzle 336 and to emerge therefrom atomized. This does not happen until a sufficient actuating pressure has built up. Shortly after the actuator has pressed the ball fully into place the stop surfaces 353 come into contact with the top of the plunger plug 331 to force the plunger plug into the media reservoir with discharge of the medium.

FIG. 7 shows still another embodiment of a discharge device 400 that can be used with the adapter 50 (FIG. 3) of the invention. The discharge device 400 has a housing 405, a finger rest 408, and a substantially cylindrical reception space 407.

On a side remote from the application area, the reception space 407 is at least partly sealed by a pressure sleeve 404 displaceably fitted in said reception space 407. At its end directed towards the application part, the pressure sleeve 404 has a stop collar 420. On an end of the pressure sleeve 404 facing the application part is provided a centering cone 433, which on fitting the pressure sleeve slides into the reception space 407 on an insertion bevel 422 provided there and consequently facilitates the insertion of the pressure sleeve 404. The pressure sleeve 404 is held in an inoperative position by means of a helical spring 423. A thumb support 424 is provided at an end of the device 400.

A pressure cone 444 of the pressure sleeve 404 surrounds a substantially cylindrical feed sleeve 409 and is positively connected therewith on a starting locking area 432 in the form of a circumferential collar in the direction of the discharge stroke A4. On applying a compressive force on the thumb support 424, the starting locking area 432 permits a force transfer from the pressure sleeve 404 to the feed sleeve 409. Counter to the discharge stroke direction A4 along the center axis of the pressure sleeve and the basic housing in the direction of the application part, on said feed sleeve 409 is provided a pressure locking area 431 spaced from the starting locking area 432. The feed sleeve 409 is closed at the end remote from the application area by the feed sleeve base 430. Above the starting locking area 432 are provided on an outer contour of the feed sleeve 409 two spaced locking rings, as well as in each case a holding ring 434 spaced in the immediate vicinity from the given locking ring 421. In each case one locking ring 421 and a holding ring 434 form an inoperative locking area 436 and an operative locking area 437 of the feed sleeve 409.

The feed sleeve 409 is provided with a cylindrical bore extended over virtually the entire length and whose diameter in the vicinity of the starting locking area 432 is reduced up to just before the feed sleeve base 430. A guide nose 446 is provided on an inner wall of the feed sleeve 409 in rotationally symmetrical circular manner and displaced by 120 degrees. In addition, a circumferential guide collar 445 is provided in the end of the feed sleeve 409 facing the application area.

On the guide collar 445 and guide noses 446, during a discharge stroke, slides a medium reservoir 402 which is zonally surrounded by the feed sleeve 409. The medium reservoir is closed with a sealing element 410, made from an elastomer.

On a piston pressure face 416 facing the application area, during the discharge stroke, the sealing element 410 is subject to the action of a compressive force by a rod or ram pressure face 415 of a pump rod or ram 403, so that the sealing element 410 displaceably fitted in the medium reservoir 402 gives rise to a counterpressure in the stored medium 417. The pump ram 403 is guided in a ram receptacle 412 of the basic housing 405, has a substantially cylindrical and rotationally symmetrical design and has close to its axis of symmetry a medium channel 406 in the form of a medium tube 411. The medium channel 406 is closed in the direction of the application part and in the inoperative state by a valve block 438 provided in a valve chamber 439 of the basic housing 405. A discharge nozzle 440 is provided above the valve block 438 in the area surrounding an outlet of the valve chamber 439 and as a result of a significant cross-sectional difference between the diameter of the discharge nozzle 440 and the surrounding area brings about a nebulization of the discharged medium 417.

An end face of the medium tube 411 remote from the application area is cut in wedge-shaped manner and consequently forms a cutting tip 414. During the discharge stroke the cutting tip 414 slides into the bore provided in the sealing element 410 and cuts through the latter at this location. As a result the medium tube 411 can be immersed in the medium 417 stored in the medium reservoir 402.

To prevent an undesired operation of the discharge device 400, between an outer contour 435 of the feed sleeve 409 acting as a working area and a corrugated element 435, there are three locking elements 419 fitted at an angle of in each case 120 degrees in circular manner around the symmetry axis of the discharge device and which positively engage in the inoperative locking area 436 of the feed sleeve 409. In addition, there is a helical spring 423, acting as a compression spring, in the reception space 407 and in a working space formed by the pressure sleeve and presses the latter positively against a circumferential stop edge 418 formed by the outer wall of the reception space 407.

To bring about a medium discharge from the discharge device 400, the user preferably places the middle and index fingers on the finger support 408, whilst simultaneously exerting a pressure on the thumb rest 424 using the thumb. The user must at least apply an actuating force which in particular consists of a biasing of the helical spring 423 and the deformation force necessary for overcoming the positive connection between the corrugated element 425 and the working area 435 of the feed sleeve 409, as well as a frictional part. As soon as the operating force is exerted by the user on the discharge device, the locking elements 419 of the corrugated element 425 are unlocked by elastic deformation of corrugated projections (not shown) located within the corrugated element 425 and allow a sliding of the working area 435 in the direction of the application area. Both the pressure sleeve 404 and the feed sleeve 409 start to move and the user only has to work against the spring tension of helical spring 423 and a sliding frictional force.

As soon as a pressure collar 429 of the feed sleeve 409 comes into contact with a medium reservoir base 428, a positive engagement is brought about therebetween the medium reservoir 402 and feed sleeve 409, which brings about a joint advance of these two components. Through the movement of the medium reservoir 402 in the discharge stroke direction A4, the sealing element 410 comes into contact with the cutting tip 414 of the medium tube 411 and is perforated when the user exerts increasing pressure on the thumb rest 424. As a result the medium tube 411 enters the medium reservoir 402 and the medium enclosed therein can initially flow in almost pressureless manner into the medium channel 406. A pressurizing of the medium 417 takes place during continuing movement of the medium reservoir

402 in the discharge stroke direction A4. The ram pressure face 415 of the pump ram 403 engages on the piston pressure face 416 of the sealing element 410 and places the medium 417 increasingly under pressure by the thumb pressure applied by the user. As a result of the rising pressure in the medium reservoir 402, the medium 417 flows along the medium channel 406 in the direction of the valve block 438, which upwardly terminates the medium channel 406. On reaching a design-predetermined minimum pressure, the valve formed by the valve block 438 and valve block 439 opens. The medium 417 is passed into the environment in nebulized form as a result of the rapid cross-sectional change in the discharge nozzle 440.

The discharge stroke is ended as soon as the front face facing the application area of the pressure sleeve 404 strikes against the front face of the corrugated element 425. The working area 435 of the feed sleeve is designed in such a way that when the locking elements 419 of the corrugated element 425 reach such a position positively engage in the operative locking area 437 and retain the feed sleeve in this position. As a further medium flow is prevented by reaching a block length of the pressure sleeve 404, the user will reduce the thumb pressure on the thumb rest 424 and the feed sleeve base 430. Thus, the energy stored in the helical spring 423 leads to a movement of the pressure sleeve 404 counter to the discharge stroke direction A4. The zonally slotted pressure cone 444 slides out of the original starting locking area 432 into the pressure locking area 431 and engages again with its stop collar 420 on the stop edge 418 of the outer sleeve of the reception space 407. This permits a further operation of the feed sleeve 409 by the pressure sleeve 404, which allows a second discharge process on applying a corresponding operating force.

It can be appreciated that the adaptor 50 of the present invention can be used with any of the spray discharge devices discussed above and with other spray discharge devices that are known in the art.

While this disclosure has been described as having exemplary designs, the present disclosure can be further modified within the spirit and scope of this disclosure. This application is, therefore, intended to cover any variations, uses, or adaptations of the disclosure using its general principles. Further, this application is intended to cover such departures from the present disclosure as come within known or customary practice in the art to which this disclosure pertains and which fall within the limits of the appended claims.

The invention claimed is:

1. An adapter for delivering a metered dose of flowable media comprising:
a spray pump device having a first end defining a luer tip and a second end configured for accessing a media reservoir to receive a metered dose of flowable media, wherein the luer tip is configured to be secured to a needle cannula to deliver the metered dose of the flowable media received from the spray pump device.

2. A device for delivering a metered dose of flowable media comprising:
a media reservoir configured for holding a flowable media;
an actuator having a first end, a second end, and a conduit extending between the first and second end, said second end of said actuator configured for movement between a first positon and a second position upon the application of an actuating force thereto;
an inlet provided at the second end of the actuator, said inlet configured for transferring a metered dose of the flowable media from the media reservoir into the conduit of the actuator;
an opening in the first end of the actuator; and
a connecting member associated with the first end of the actuator, said connecting member configured for cooperation with a needle cannula such that the needle cannula is in fluid communication with the opening in the first end of the actuator to deliver the metered dose of the flowable media.

3. The device of claim 2, wherein the connecting member comprises a luer tip.

4. The device of claim 2, wherein the needle cannula is preassembled to the luer tip and the device further includes a removable cover for the needle cannula.

5. The device of claim 2, wherein the media reservoir is a vial and the second end of the actuator is configured for attachment to the vial.

6. The device of claim 5, wherein the vial includes a pierceable septum and the second end of the actuator includes a piercing element associated therewith, said piercing element including a fluid passageway for providing fluid communication between the actuator and vial.

7. The device of claim 6, wherein the piercing element comprises a non-patient cannula.

8. The device of claim 2, wherein the actuator includes a biasing element to return the actuator to the first positon after application of the actuating force thereto.

9. The device of claim 1, including a valve configured for cooperation with the inlet of the actuator, wherein movement of the actuator to the second position causes said valve to open to admit the metered dose of the flowable media into the conduit of the actuator.

10. The device of claim 1, wherein the dose of flowable media is applied subcutaneously or intramuscularly.

11. A method of delivering a metered dose of flowable media comprising:
providing a media reservoir having a spray pump device associated therewith, the spray pump device having an exit opening and being configured for supplying a metered dose upon application of an actuation force thereto;
securing a needle cannula to the exit opening of the spray pump device, wherein application of the actuation force to the spray pump device causes said metered dose of flowable media to exit through the needle cannula.

12. The method of claim 11, including providing a luer tip to the exit opening of the spray pump device for securing the needle cannula thereto.

13. The method of claim 11, wherein the media reservoir comprises a vial having a pierceable septum and the spray pump device includes a piercing element configured for piercing the septum and wherein the method includes piercing the septum of the vial with the piercing element.

14. The method of claim 13, wherein the piercing element comprises a non-patient cannula.

15. The method of claim 13, wherein at least one of the vial and the spray pump device includes a connecting assembly to secure the vial and the spray pump device together.

16. The method of claim 13, including providing a removable cover for the needle cannula and a collapsible sleeve for the piercing element.

17. The method of claim 13, wherein the dose of flowable media is applied subcutaneously or intramuscularly.
